(19) United States
(12) Patent Application Publication
(10) Pub. No.: US 2026/0165678 A1
(43) Pub. Date: Jun. 18, 2026

(54) ULTRASOUND DIAGNOSTIC SYSTEM AND CONTROL METHOD OF ULTRASOUND DIAGNOSTIC SYSTEM

(71) Applicant: FUJIFILM Corporation, Tokyo (JP)

(72) Inventor: Riki IGARASHI, Kanagawa (JP)

(73) Assignee: FUJIFILM Corporation, Tokyo (JP)

(21) Appl. No.: 19/406,983

(22) Filed: Dec. 3, 2025

(30) Foreign Application Priority Data
Dec. 16, 2024 (JP) 2024-219664

(51) Int. Cl. A61B 8/00; A61B 90/00

(52) U.S. Cl. CPC A61B 8/4245; A61B 90/39; A61B 8/461; A61B 2090/3937

(57) ABSTRACT

Provided are an ultrasound diagnostic system and a control method of the ultrasound diagnostic system capable of estimating a position and an orientation of an ultrasound probe even in a case where the ultrasound probe is in a blind spot of a visual field of the optical camera.

An ultrasound diagnostic system includes an ultrasound probe having a reference figure projection light source that projects a reference figure, a projection target member onto which an image of the reference figure is projected from the reference figure projection light source, a diagnostic apparatus connected to the ultrasound probe, and an optical camera that is connected to the diagnostic apparatus and acquires an optical image in which the projection target member appears, in which the diagnostic apparatus includes a probe position estimation unit that estimates a position and an orientation of the ultrasound probe based on the image of the reference figure projected onto the projection target member that appears in the optical image.

### CROSS-REFERENCE TO RELATED APPLICATIONS

The present application claims priority under 35 U.S.C. § 119 to Japanese Patent Application No. 2024-219664, filed on Dec. 16, 2024. The above application is hereby expressly incorporated by reference, in its entirety, into the present application.

### BACKGROUND OF THE INVENTION

### 1. Field of the Invention

The present invention relates to an ultrasound diagnostic system and a control method of the ultrasound diagnostic system, for estimating a position and an orientation of an ultrasound probe.

### 2. Description of the Related Art

In the related art, an ultrasound examination of a subject is performed by capturing an ultrasound image representing a tomographic plane in the subject by using a so-called ultrasound probe. In this case, for example, a position and an orientation of the ultrasound probe may be measured, for example, so that a user such as a doctor confirms the position and orientation of the ultrasound probe corresponding to the captured ultrasound image after examining the subject. As one of the methods of measuring the position and the orientation of the ultrasound probe, for example, as disclosed in JP2016-083022A, a technique of measuring a position and an orientation of the ultrasound probe by disposing a marker on the ultrasound probe and imaging the marker with an optical camera is known. The method of measuring the position and the orientation of the ultrasound probe using the marker has an advantage that the required equipment is inexpensive and can be easily installed, as compared with, for example, a method of measuring the position and the orientation of the ultrasound probe by using a so-called magnetic sensor or the like.

### SUMMARY OF THE INVENTION

However, there is a case where a marker disposed on the ultrasound probe during the ultrasound examination of the subject is covered by the body or the like of the subject and enters a blind spot of the visual field of the optical camera. In this case, since the marker cannot be captured by the optical camera, there is a problem that it is difficult to measure the position and the orientation of the ultrasound probe using the technique disclosed in JP2016-083022A.

The present invention has been made in order to solve such a problem in the related art, and an object of the present invention is to provide an ultrasound diagnostic system and a control method of the ultrasound diagnostic system capable of estimating a position and an orientation of an ultrasound probe even in a case where the ultrasound probe is in a blind spot of the visual field of the optical camera.

The above object can be achieved with the following configurations.

[1] An ultrasound diagnostic system comprising an ultrasound probe having a reference figure projection light source that projects a reference figure, a projection target member onto which an image of the reference figure is projected from the reference figure projection light source, a diagnostic apparatus connected to the ultrasound probe, and an optical camera that is connected to the diagnostic apparatus and acquires an optical image in which the projection target member appears, in which the diagnostic apparatus includes a probe position estimation unit that estimates a position and an orientation of the ultrasound probe based on the image of the reference figure projected onto the projection target member that appears in the optical image.

[2] The ultrasound diagnostic system according to [1], in which a marker is installed on the ultrasound probe, and the probe position estimation unit further estimates the position and the orientation of the ultrasound probe by taking into account the marker installed on the ultrasound probe that appears in the optical image.

[3] The ultrasound diagnostic system according to [2], further comprising a blind spot state determination unit that determines whether or not the marker of the ultrasound probe enters a blind spot of a visual field of the optical camera by analyzing the optical image acquired by the optical camera, in which the probe position estimation unit estimates the position and the orientation of the ultrasound probe based only on the image of the reference figure projected onto the projection target member while the blind spot state determination unit determines that the marker has entered the blind spot of the optical camera.

[4] The ultrasound diagnostic system according to [3], in which the blind spot state determination unit determines that the ultrasound probe has entered the blind spot of the optical camera in a case where a state in which the marker installed on the ultrasound probe is not read from the optical image continues for a predetermined time or longer.

[5] The ultrasound diagnostic system according to [3] or [4], in which the reference figure projection light source stops the projection of the reference figure onto the projection target member in a case where the blind spot state determination unit determines that the marker is located outside the blind spot of the optical camera, and starts the projection of the reference figure onto the projection target member in a case where the blind spot state determination unit determines that the marker has entered the blind spot of the optical camera.

[6] The ultrasound diagnostic system according to any one of [1] to [5], further comprising a memory, in which the diagnostic apparatus includes an image acquisition unit that acquires an ultrasound image of a subject by transmitting and receiving an ultrasound beam using the ultrasound probe, and the probe position estimation unit stores, in the memory, information on the estimated position and orientation of the ultrasound probe and the ultrasound image in association with each other.

[7] A control method of an ultrasound diagnostic system, the control method comprising: projecting a reference figure onto a projection target member from an ultrasound probe; acquiring an optical image in which the projection target member appears by using an optical camera; and estimating a position and an orientation of the ultrasound probe based on an image of the reference figure projected onto the projection target member that appears in the optical image.

The present invention provides an ultrasound diagnostic system that comprises an ultrasound probe having a reference figure projection light source that projects a reference figure, a projection target member onto which an image of the reference figure is projected from the reference figure projection light source, a diagnostic apparatus connected to the ultrasound probe, and an optical camera that is connected to the diagnostic apparatus and acquires an optical image in which the projection target member appears, in which the diagnostic apparatus includes a probe position estimation unit that estimates a position and an orientation of the ultrasound probe based on the image of the reference figure projected onto the projection target member that appears in the optical image, so that the position and the orientation of the ultrasound probe can be estimated even in a case where the ultrasound probe is in a blind spot of a visual field of the optical camera.

### BRIEF DESCRIPTION OF THE DRAWINGS

FIG. 1 is a block diagram illustrating a configuration of an ultrasound diagnostic system according to a first embodiment of the present invention.

FIG. 2 is a block diagram illustrating an internal configuration of an ultrasound probe and a diagnostic apparatus in the first embodiment of the present invention.

FIG. 3 is a block diagram illustrating an internal configuration of a transmission and reception circuit in the first embodiment of the present invention.

FIG. 4 is a block diagram illustrating an internal configuration of an image generation unit in the first embodiment of the present invention.

FIG. 5 is a diagram schematically illustrating an example of an appearance of the ultrasound probe in the first embodiment of the present invention.

FIG. 6 is a diagram schematically illustrating an example of a projection target member in the first embodiment of the present invention.

FIG. 7 is a diagram schematically illustrating an example of an optical image in which the projection target member and the ultrasound probe appear.

FIG. 8 is a diagram schematically illustrating an example of an AR display image in which a virtual display is added to the optical image.

FIG. 9 is a flowchart illustrating an operation of the ultrasound diagnostic system according to the first embodiment of the present invention.

FIG. 10 is a diagram schematically illustrating a modification example of an appearance of the ultrasound probe in the first embodiment of the present invention.

FIG. 11 is a block diagram illustrating an internal configuration of an ultrasound probe and a diagnostic apparatus in a second embodiment of the present invention.

### DESCRIPTION OF THE PREFERRED EMBODIMENTS

Hereinafter, embodiments of the present invention will be described with reference to the accompanying drawings.

The description of configuration requirements described below is given based on the representative embodiment of the present invention, but the present invention is not limited to such an embodiment.

In the present specification, a numerical range represented using “to” means a range including the numerical values before and after “to” as a lower limit value and an upper limit value.

In the present specification, the terms “same” and “identical” include an error range that is generally allowed in the technical field.

### First Embodiment

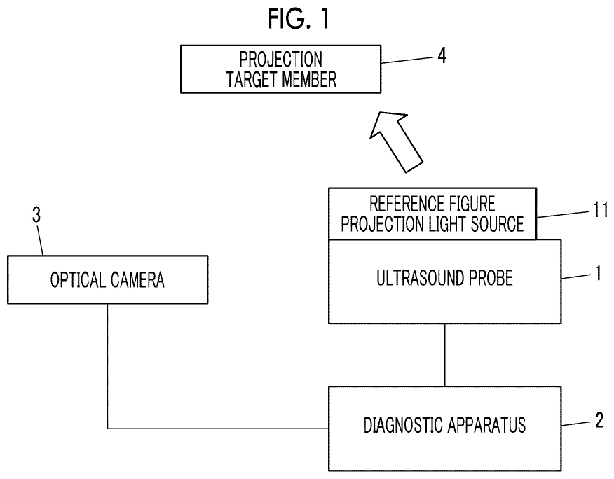

FIG. 1 illustrates a configuration of an ultrasound diagnostic system according to a first embodiment of the present invention. The ultrasound diagnostic system comprises an ultrasound probe 1, a diagnostic apparatus 2 connected to the ultrasound probe 1, an optical camera 3 connected to the diagnostic apparatus 2, and a projection target member 4 consisting of a plane shape such as a so-called screen. The ultrasound probe 1 has a reference figure projection light source 11 that projects a reference figure, which will be described later, on the projection target member 4.

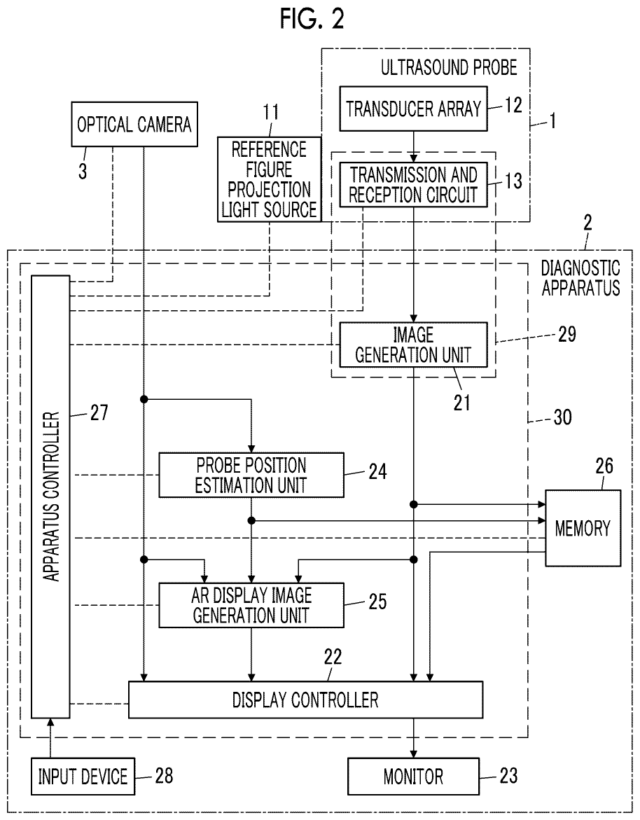

FIG. 2 illustrates an internal configuration of the ultrasound probe 1 and the diagnostic apparatus 2. The ultrasound probe 1 and the diagnostic apparatus 2 are connected to each other via so-called wired communication or so-called wireless communication. The ultrasound probe 1 comprises a transducer array 12 and a transmission and reception circuit 13 connected to the transducer array 12.

The diagnostic apparatus 2 comprises an image generation unit 21 connected to the transmission and reception circuit 13. In the diagnostic apparatus 2, a display controller 22 and a monitor 23 are sequentially connected to the image generation unit 21. Further, the diagnostic apparatus 2 comprises a probe position estimation unit 24 connected to the optical camera 3. An augmented reality (AR) display image generation unit 25 is connected to the optical camera 3, the image generation unit 21, and the probe position estimation unit 24. Further, a memory 26 is connected to the image generation unit 21 and the probe position estimation unit 24. The optical camera 3, the AR display image generation unit 25, and the memory 26 are connected to the display controller 22. In addition, an apparatus controller 27 is connected to the optical camera 3, the reference figure projection light source 11 and the transmission and reception circuit 13 of the ultrasound probe 1, the image generation unit 21, the display controller 22, the probe position estimation unit 24, the AR display image generation unit 25, and the memory 26. An input device 28 is connected to the apparatus controller 27.

The transmission and reception circuit 13 and the image generation unit 21 constitute an image acquisition unit 29. Further, the image generation unit 21, the display controller 22, the probe position estimation unit 24, the AR display image generation unit 25, and the apparatus controller 27 constitute a processor 30 for the diagnostic apparatus 2.

The ultrasound probe 1 is used to capture a so-called ultrasound image that represents a tomographic plane inside a subject by transmitting an ultrasound beam into the subject and receiving an ultrasound echo reflected from the inside of the subject in a state of being in contact with a body surface of the subject.

The transducer array 12 of the ultrasound probe 1 includes a plurality of ultrasound transducers that are one-dimensionally or two-dimensionally arranged. According to a drive signal supplied from the transmission and reception circuit 13, each of the ultrasound transducers transmits an ultrasound wave and receives the ultrasound echo from the subject to output a signal based on the ultrasound echo. For example, each ultrasound transducer is configured by forming electrodes at both ends of a piezoelectric body consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), or the like.

The image acquisition unit 29 configured by the transmission and reception circuit 13 and the image generation unit 21 acquires an ultrasound image by transmitting and receiving the ultrasound beam by using the ultrasound probe 1.

The transmission and reception circuit 13 transmits the ultrasound wave from the transducer array 12 and generates a sound ray signal based on the received signal acquired by the transducer array 12 under control of the apparatus controller 27. As illustrated in FIG. 2, the transmission and reception circuit 13 includes a pulser 41 connected to the transducer array 12, and an amplifying unit 42, an analog-to-digital (AD) conversion unit 43, and a beam former 44 that are sequentially connected in series to the transducer array 12.

The pulser 41 includes, for example, a plurality of pulse generators, adjusts a delay amount of each drive signal, based on a transmission delay pattern selected in accordance with a control signal from the apparatus controller 27, so that the ultrasound waves transmitted from the plurality of ultrasound transducers of the transducer array 12 form an ultrasound beam, and supplies each drive signal to the plurality of ultrasound transducers. In this way, in a case where a pulsed or continuous-wave voltage is applied to the electrodes of the ultrasound transducers of the transducer array 12, a piezoelectric body expands and contracts to generate pulsed or continuous-wave ultrasound waves from each ultrasound transducer, thereby forming an ultrasound beam from the combined wave of these ultrasound waves.

The transmitted ultrasound beam is reflected by a target, for example, a site of the subject, and propagates toward the transducer array 12 of the ultrasound probe 1. The ultrasound echo propagating toward the transducer array 12 in this manner is received by each ultrasound transducer constituting the transducer array 12. In this case, each ultrasound transducer constituting the transducer array 12 expands and contracts by receiving the propagating ultrasound echo to generate a reception signal, which is an electric signal, and outputs the reception signal to the amplifying unit 42.

The amplifying unit 42 amplifies the signals input from each ultrasound transducer constituting the transducer array 12, and transmits the amplified signals to the AD conversion unit 43. The AD conversion unit 43 converts the signals transmitted from the amplifying unit 42 into digital reception data. The beam former 44 performs so-called receive focusing processing by applying respective delays to the received data from the AD conversion unit 43 and summing them. With the receive focusing processing, the respective received data converted by the AD conversion unit 43 is summed in phase, and a sound ray signal in which a focus of the ultrasound echo is narrowed is acquired.

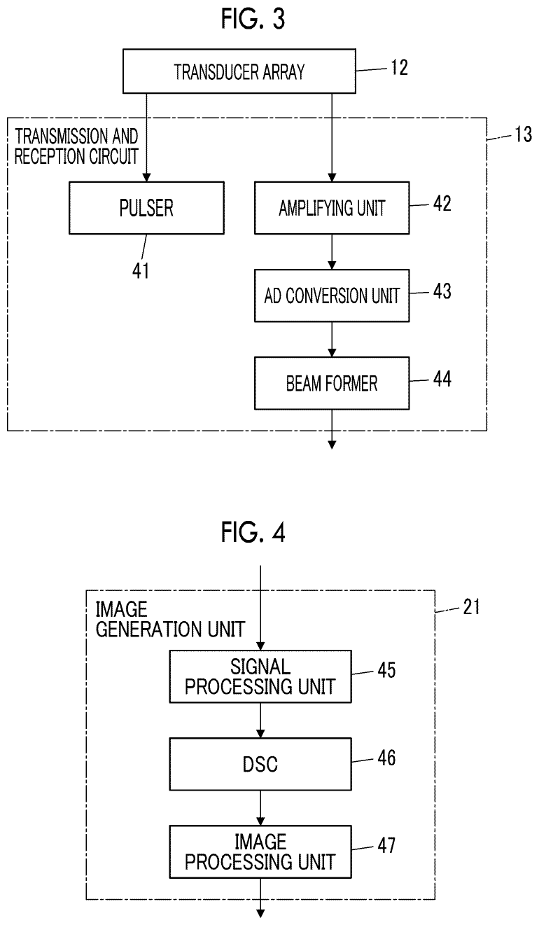

As illustrated in FIG. 3, the image generation unit 21 has a configuration in which a signal processing unit 45, a digital scan converter (DSC) 46, and an image processing unit 47 are sequentially connected in series.

The signal processing unit 45 generates a B-mode image signal, which is tomographic image information related to tissues inside the subject, by performing, on the sound ray signal received from the transmission and reception circuit 13, correction of the attenuation due to a distance in accordance with a depth of a reflection position of the ultrasound by using a sound velocity value set by the apparatus controller 27, and then performing envelope detection processing.

The DSC 46 converts (raster-converts) the B-mode image signal generated in the signal processing unit 45 into an image signal conforming to a normal television signal scanning method.

The image processing unit 47 performs various types of necessary image processing such as gradation processing on the B-mode image signal input from the DSC 46, and then transmits the B-mode image signal to the display controller 22, the AR display image generation unit 25, and the memory 26. Hereinafter, the B-mode image signal subjected to the image processing by the image processing unit 47, will be referred to as an ultrasound image.

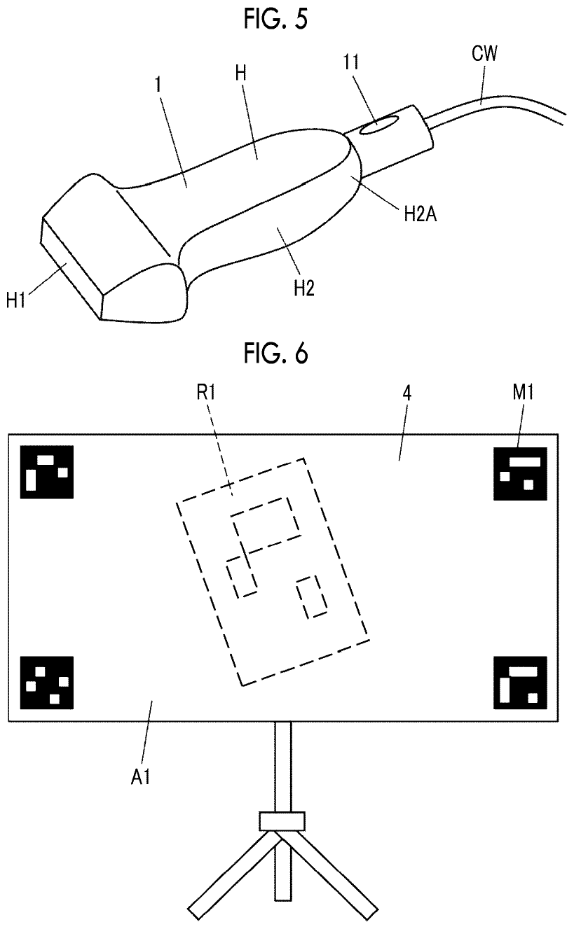

Here, the ultrasound probe 1 has, for example, a housing H as illustrated in FIG. 5. The transducer array 12 and the transmission and reception circuit 13 are accommodated in the housing H. The housing H has an array accommodation portion H1 that is a portion in contact with the body surface of the subject and that accommodates the transducer array 12, and a grip portion H2 that is gripped by a user such as a doctor to hold the ultrasound probe 1. The reference figure projection light source 11 can be attached to, for example, a tip portion H2A of the grip portion H2 that is located on an opposite side to the array accommodation portion H1. In FIG. 5, as an example, the ultrasound probe 1 is depicted as a so-called wired probe connected to the diagnostic apparatus 2 via a connection cable CW.

The reference figure projection light source 11 is a light source for projecting a reference figure, which can be used as a so-called AR marker such as Augmented Reality University of Cordoba (ArUco), toward the projection target member 4. The reference figure projection light source 11 can be configured by, for example, a small so-called projector or projection apparatus.

The projection target member 4 is, for example, as illustrated in FIG. 6, a member having a planar shape and having a projection target region A1 that does not have a pattern. An image R1 of the reference figure, projected by the reference figure projection light source 11, appears in the projection target region A1. The projection target member 4 can have a marker M1 that can be used as an AR marker such as the ArUco. The marker M1 is different from the reference figure projected onto the projection target member 4 from the reference figure projection light source 11. The type of the projection target member 4 is not particularly limited as long as the projection target member 4 has the projection target region A1, and the projection target member 4 may be, for example, a screen as illustrated in FIG. 6 or a wall or a ceiling of an examination room.

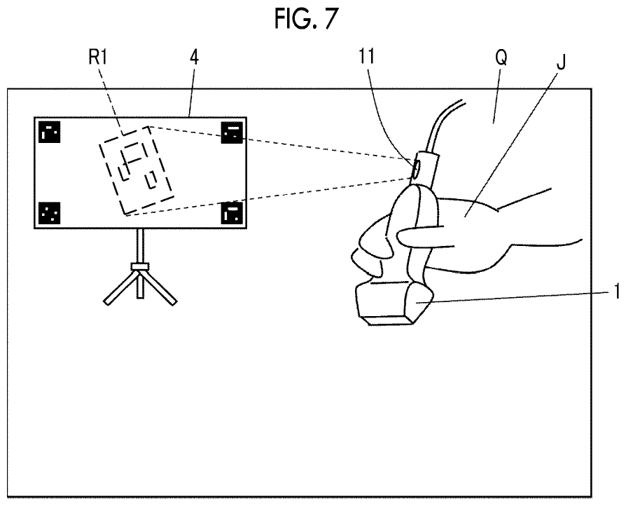

The optical camera 3 acquires an optical image in which the projection target member 4 appears. The optical camera 3 includes, for example, an image sensor such as a so-called charge coupled device (CCD) image sensor or a so-called complementary metal-oxide-semiconductor (CMOS) image sensor, and acquires an optical image Q obtained by optically imaging the ultrasound probe 1 and the projection target member 4 under control of the apparatus controller 27, for example, as illustrated in FIG. 7. In the example of FIG. 7, the optical image Q in which the ultrasound probe 1 gripped by a hand J of a user and the projection target member 4 on which the image R1 of the reference figure is projected by the reference figure projection light source 11 attached to the ultrasound probe 1 are illustrated. The optical camera 3 can be fixedly disposed at a position where the ultrasound probe 1 and the projection target member 4 can be clearly imaged.

The probe position estimation unit 24 estimates a position and an orientation of the ultrasound probe 1 based on the image R1 of the reference figure, which is projected onto the projection target member 4 and appears in the optical image Q acquired by the optical camera 3. In this case, the probe position estimation unit 24 first reads the marker M1 disposed on the projection target member 4 and estimates the position and the orientation of the projection target member 4 with respect to the optical camera 3. Next, the probe position estimation unit 24 reads the image R1 of the reference figure projected onto the projection target member 4 and estimates the position and the orientation of the ultrasound probe 1 with respect to the projection target member 4. Finally, the probe position estimation unit 24 can estimate the position and the orientation of the ultrasound probe 1 with respect to the optical camera 3, based on information on the position and the orientation of the projection target member 4 with respect to the optical camera 3 and information on the position and the orientation of the ultrasound probe 1 with respect to the projection target member 4.

Here, since the position and the orientation of the projection target member 4 with respect to the optical camera 3 are fixed, the probe position estimation unit 24 can also store the position and orientation of the projection target member 4 with respect to the optical camera 3 in advance. In this case, the probe position estimation unit 24 can estimate the position and the orientation of the ultrasound probe 1 with respect to the optical camera 3, based on the stored information on the position and the orientation of the projection target member 4 with respect to the optical camera 3 without reading the marker M1 disposed on the projection target member 4, and the information on the position and the orientation of the ultrasound probe 1 with respect to the projection target member 4 estimated by reading the image R1 of the reference figure projected onto the projection target member 4.

The probe position estimation unit 24 can read the marker M1 of the projection target member 4 and the image R1 of the reference figure by using a known algorithm for reading the figure used as the AR marker. For example, in a case where the marker M1 of the projection target member 4 and the image R1 of the reference figure represent ArUco, the marker M1 of the projection target member 4 and the image R1 of the reference figure can be read by using an algorithm for ArUco included in OpenCV (registered trademark), which is a library.

In general, in a case of reading a figure used as an AR marker and estimating a distance between the optical camera 3 and the figure, so-called internal parameters including a focal length of a lens of the optical camera 3, an optical center, and a shear coefficient, so-called external parameters representing the degree of freedom of rotation and translation of the optical camera 3, and a distortion coefficient of the lens are required. The probe position estimation unit 24 acquires internal parameters, external parameters, and distortion coefficients from the optical camera 3, before estimating the position and the orientation of the ultrasound probe 1, and estimates the position and the orientation of the ultrasound probe 1 with respect to the optical camera 3 from the acquired internal parameters, external parameters, and distortion coefficients, and reading results of the marker M1 of the projection target member 4 and the image R1 of the reference figure.

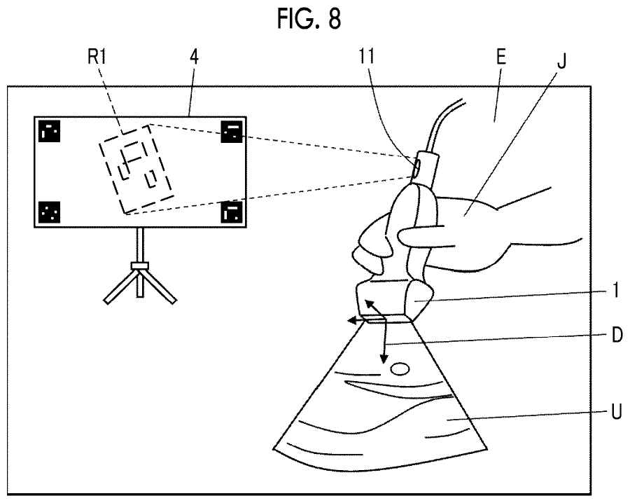

The AR display image generation unit 25 generates an AR display image E as illustrated in FIG. 8, for example, based on the optical image Q acquired by the optical camera 3, the ultrasound image U acquired by the image acquisition unit 29, and the information on the position and the orientation of the ultrasound probe 1 that is estimated by the probe position estimation unit 24, and displays the AR display image E on the monitor 23 via the display controller 22. In the example of FIG. 8, the AR display image E in which the ultrasound probe 1 gripped by a hand J of a user and the projection target member 4 on which the image R1 of the reference figure is projected by the reference figure projection light source 11 attached to the ultrasound probe 1 are illustrated. In the AR display image E, a direction vector D in three directions representing the orientation of the ultrasound probe 1 and the ultrasound image U displayed to extend from the tip of the ultrasound probe 1 is virtually illustrated. The user can easily visually recognize the orientation of the ultrasound probe 1 with respect to the subject and a positional relationship between the acquired ultrasound image U and the subject by confirming the image virtually illustrated in this way.

The memory 26 stores the ultrasound image U acquired by the image acquisition unit 29 and the information on the position and the orientation of the ultrasound probe 1 estimated by the probe position estimation unit 24 in association with each other. A user who is not skilled in interpreting the ultrasound image U may have difficulty in understanding which part of the subject is represented by the ultrasound image U, but the user can easily understand which part of the subject is represented by the ultrasound image U by confirming the information on the position and the orientation of the ultrasound probe 1 stored in correspondence with the ultrasound image U.

As the memory 26, for example, recording media such as a flash memory, a hard disk drive (HDD), a solid state drive (SSD), a flexible disk (FD), a magneto-optical disk (MO disk), a magnetic tape (MT), a random access memory (RAM), a compact disc (CD), a digital versatile disc (DVD), a secure digital card (SD card), or a universal serial bus memory (USB memory) can be used.

The display controller 22 performs predetermined processing on the ultrasound image U acquired by the image acquisition unit 29, the AR display image E generated by the AR display image generation unit 25, and the like, and displays the ultrasound image U, the AR display image E, and the like on the monitor 23, under the control of the apparatus controller 27.

The monitor 23 displays the ultrasound image U or the like, under the control of the display controller 22, and includes, for example, a display device such as a liquid crystal display (LCD) or an organic electroluminescence display (organic EL display).

The apparatus controller 27 controls each unit of the diagnostic apparatus 2, the transmission and reception circuit 13 of the ultrasound probe 1, the reference figure projection light source 11, and the optical camera 3, based on a control program and the like stored in advance.

The input device 28 is an input device for the user to perform an input operation and includes, for example, a device such as a keyboard, a mouse, a track ball, a touch pad, and a touch sensor disposed on the monitor 23 in a superimposed manner.

In the present embodiment, each processing is executed by any computer. In addition, any computer may execute these types of processing by the processor 30 as hardware, a program as software, or a combination thereof. In such a case, the processor 30 is configured to execute various types of processing in the present embodiment in cooperation with the program, and may function as each unit or each means in the present embodiment. In addition, the execution order of the processing by the processor 30 is not limited to the above order and may be changed as appropriate. Any computer may be a general-purpose computer, a computer for a specific application, a workstation, or another system capable of executing each processing.

The processor 30 may be composed of one or a plurality of pieces of hardware, and types of hardware are not limited. For example, the processor 30 may be composed of hardware such as a central processing unit (CPU), a micro processing unit (MPU), a programmable logic device such as a field programmable gate array (FPGA), a dedicated circuit for executing specific processing, such as an application specific integrated circuit (ASIC), a graphic processing unit (GPU), or a neural processing unit (NPU). Furthermore, the types of hardware may be a combination of different types of hardware. In a case in which the plurality of types of hardware are configured to execute one or a plurality of types of processing of the processor 30, the plurality of types of hardware may be present in devices physically separated from each other or may be present in the same device. Furthermore, in any of the embodiments, the order of each processing performed by the processor 30 is not limited to the above order, and may be changed as appropriate. The hardware is composed of an electric circuit (circuitry) in which circuit elements such as semiconductor elements are combined.

Further, the program may be software, such as firmware or a microcode. Furthermore, the program may be, for example, a program module group, and each function thereof may be implemented by the processor 30 configured to execute each function. The program may be a program code or a plurality of code segments stored in one or a plurality of non-transitory computer-readable media (for example, a storage medium and other storages). The program may be divided and stored in a plurality of non-transitory computer-readable media present in apparatuses physically separated from each other. The program code or the code segment may represent any combination of procedures, functions, subprograms, routines, subroutines, modules, software packages, classes, instructions, data structures, or program statements. The program code or the code segment may be connected to another code segment or a hardware circuit by transmitting and receiving information, data, an argument, a parameter, or contents of a memory.

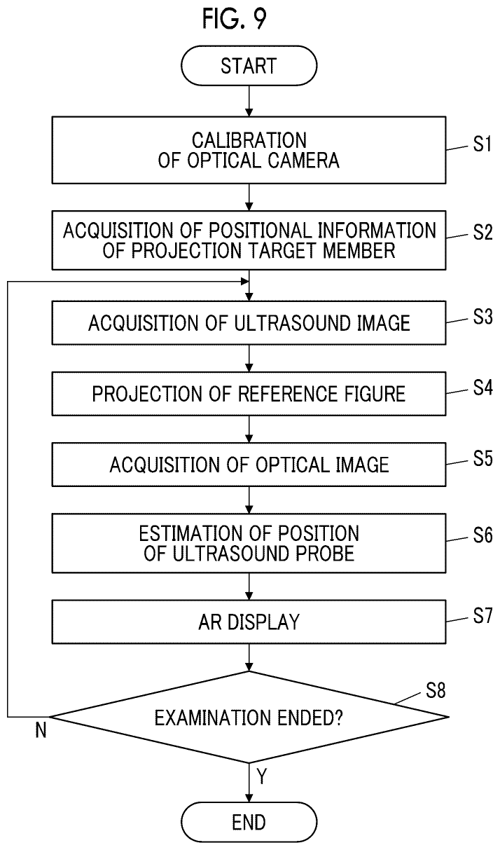

Hereinafter, an operation of the ultrasound diagnostic apparatus according to the embodiment will be described with reference to a flowchart illustrated in FIG. 9. The optical camera 3 is disposed at a position where the ultrasound probe 1 and the projection target member 4 enter field of view of the optical camera 3.

In Step S1, the apparatus controller 27 performs calibration of the optical camera 3. Through this calibration, the internal parameters, the external parameters, and the distortion coefficient of the optical camera 3 can be acquired. The apparatus controller 27 can perform the calibration of the optical camera 3 by using, for example, a method described in Z. Zhang, “A flexible new technique for camera calibration”, IEEE Transactions on Pattern Analysis and Machine Intelligence, 22(11):1330-1334, 2000.

In Step S2, positional information including a position and an orientation of the projection target member 4 with respect to the optical camera 3 is acquired. For example, in a case where the marker M1 is disposed on the projection target member 4 as illustrated in FIG. 6, an optical image Q in which the marker M1 of the projection target member 4 appears is acquired by the optical camera 3 under the control of the apparatus controller 27, and the marker M1 appearing in the optical image Q is read by the probe position estimation unit 24, so that the positional information of the projection target member 4 with respect to the optical camera 3 is acquired. In addition, in a case where the positional relationship between the optical camera 3 and the projection target member 4 is stored in advance, the probe position estimation unit 24 can acquire the positional information of the projection target member 4 with respect to the optical camera 3 that is stored in advance. In addition, the probe position estimation unit 24 can also acquire, for example, a value input by the user via the input device 28, as the positional information of the projection target member 4 with respect to the optical camera 3.

In a case where Step S2 is completed, the user such as a doctor starts the ultrasound examination of the subject in which the ultrasound image U representing the tomographic plane in the subject is captured by gripping the ultrasound probe 1. In Step S3, the user disposes the ultrasound probe 1 at a position on the body surface of the subject for capturing the ultrasound image U. The image acquisition unit 29 acquires the ultrasound image U. In such a case, under the control of the apparatus controller 27, the transmission and reception of the ultrasound from the plurality of transducers of the transducer array 12 are started in accordance with the drive signal from the pulser 41 of the transmission and reception circuit 13 of the ultrasound probe 1, the ultrasound echo from the subject is received by the plurality of transducers of the transducer array 12, and the reception signal, as the analog signal, is output to the amplifying unit 42 and is amplified, and then is subjected to the AD conversion via the AD conversion unit 43 to acquire reception data.

The receive focusing processing is performed on the reception data by the beam former 44, the sound ray signal generated thereby is transmitted to the image generation unit 21 of the diagnostic apparatus 2, and thus the ultrasound image U is generated by the image generation unit 21. In this case, the signal processing unit 45 of the image generation unit 21 performs the correction of the attenuation in accordance with the depth of a reflection position of the ultrasound and the envelope detection processing on the sound ray signal, the DSC 46 performs the conversion into the image signal in accordance with the normal television signal scanning method, and the image processing unit 47 performs various types of necessary image processing, such as gradation processing. The ultrasound image U generated in Step S3 in this way is transmitted to the display controller 22, the AR display image generation unit 25, and the memory 26.

In Step S4, the apparatus controller 27 projects the reference figure from the reference figure projection light source 11 attached to the ultrasound probe 1 toward the projection target member 4. As a result, for example, as illustrated in FIG. 6, the image R1 of the reference figure is projected onto the projection target member 4.

In Step S5, the optical camera 3 acquires the optical image Q illustrated in FIG. 7 in which the image R1 of the reference figure projected onto the projection target member 4 in Step S4 appears, under the control of the apparatus controller 27.

In Step S6, the probe position estimation unit 24 estimates a position and an orientation of the ultrasound probe 1 based on the position and the orientation of the projection target member 4 with respect to the optical camera 3 acquired in Step S2 and the image R1 of the reference figure appearing in the optical image Q acquired in Step S5. In this case, the probe position estimation unit 24 estimates the position and the orientation of the ultrasound probe 1 with respect to the projection target member 4 by reading the image R1 of the reference figure appearing in the optical image Q acquired in Step S5, and estimates the position and the orientation of the ultrasound probe 1 with respect to the optical camera 3 from the information on the position and the orientation of the projection target member 4 with respect to the optical camera 3 acquired in Step S2 and the information on the estimated position and orientation of the ultrasound probe 1 with respect to the projection target member 4.

In general, a technique of estimating the position and the orientation of the ultrasound probe 1 with respect to the optical camera 3 by reading a figure used as an AR marker disposed on the ultrasound probe 1 with the optical camera 3 is known. However, depending on the position on the subject with which the ultrasound probe 1 is brought into contact, the ultrasound probe 1 may enter a blind spot of the visual field of the optical camera 3, that is, may deviate from the visual field of the optical camera 3, and in this case, there is a problem that the position and the orientation of the ultrasound probe 1 cannot be estimated.

According to the processing of Step S6, the position and the orientation of the ultrasound probe 1 with respect to the optical camera 3 are estimated from the information on the position and orientation of the projection target member 4 with respect to the optical camera 3 acquired in Step S2 and the information on the estimated position and orientation of the ultrasound probe 1 with respect to the projection target member 4. Therefore, even in a case where the ultrasound probe 1 is in the blind spot of the field of view of the optical camera 3, the position and the orientation of the ultrasound probe 1 with respect to the optical camera 3 can be estimated.

In Step S7, the AR display image generation unit 25 generates an AR display image E as illustrated in FIG. 8, based on the ultrasound image U acquired in Step S3, the optical image Q acquired in Step S5, and the information on the position and the orientation of the ultrasound probe 1 estimated in Step S6, and displays the AR display image E on the monitor 23. The user can easily visually recognize the orientation of the ultrasound probe 1 with respect to the subject and a positional relationship between the acquired ultrasound image U and the subject by confirming the AR display image E displayed on the monitor 23.

In Step S8, the apparatus controller 27 determines whether or not to end the ultrasound examination of the subject. The apparatus controller 27 can determine to end the ultrasound examination of the subject in a case where the user inputs an instruction to end the ultrasound examination via, for example, the input device 28, and can determine to continue the ultrasound examination of the subject in a case where the user does not input a specific instruction via the input device 28.

The processing of Step S3 to Step S8 is repeated as long as it is determined to continue the ultrasound examination of the subject in Step S8. In a case where it is determined in Step S8 to end the ultrasound examination of the subject, the operation of the ultrasound diagnostic system according to the flowchart illustrated in FIG. 9 is completed.

As described above, with the ultrasound diagnostic system according to the first embodiment of the present invention, the probe position estimation unit 24 estimates the position and the orientation of the ultrasound probe 1 based on the image R1 of the reference figure projected onto the projection target member 4 appearing in the optical image Q acquired by the optical camera 3. Therefore, even in a case where the ultrasound probe 1 is in a blind spot of the optical camera 3, the position and the orientation of the ultrasound probe 1 with respect to the optical camera 3 can be estimated.

Although it has been described that the transmit and receive circuit 13 is provided in the ultrasound probe 1, the transmission and reception circuit 13 may be provided in the diagnostic apparatus 2.

Further, although it has been described that the image generation unit 21 is provided in the diagnostic apparatus 2, the image generation unit 21 may be provided in the ultrasound probe 1.

The diagnostic apparatus 2 may be a so-called stationary type, a portable type that is easy to carry, or a so-called handheld type that is configured by, for example, a smartphone or a tablet type computer. In this way, the type of equipment constituting the diagnostic apparatus 2 is not particularly limited.

Although FIG. 5 illustrates that the reference figure projection light source 11 is attached to the housing H of the ultrasound probe 1 from the outside, the attachment mode thereof is not particularly limited as long as the reference figure projection light source 11 is attached to the ultrasound probe 1. For example, a hole can be formed in the housing H, and the reference figure projection light source 11 can be built into the housing H so as to project the reference figure from the hole.

In relation to FIG. 8, it has been described that the direction vector D and the ultrasound image U are virtually described in the AR display image E, but the virtual display is not particularly limited to this. For example, the AR display image E can also include any of the direction vector D or the ultrasound image U.

In Step S7 of the flowchart of FIG. 9, the generation of the AR display image E and the display of the AR display image E on the monitor 23 have been described. However, instead of this, the ultrasound image U acquired in Step S3 and the information on the position and the orientation of the ultrasound probe 1 estimated in Step S6 can be associated with each other and stored in the memory 26. In addition, the AR display image E can be generated and displayed on the monitor 23, and the ultrasound image U and the information on the position and the orientation of the ultrasound probe 1 can be associated with each other and stored in the memory 26.

In addition, in the flowchart of FIG. 9, it has been described that the processing of Step S4 to Step S6 is performed after the processing of Step S3, but the processing of Step S3 can be performed after the processing of Step S4 to Step S6, and the processing of Step S3 and the processing of Step S4 to Step S6 can be performed at the same time.

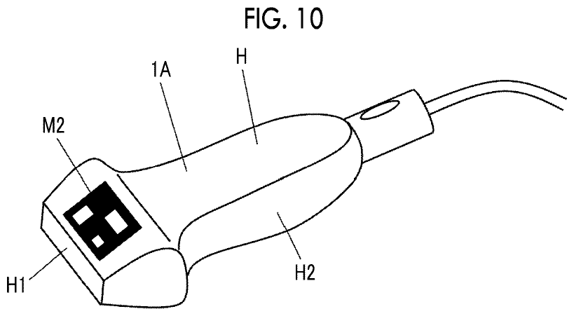

In addition, for example, as illustrated in FIG. 10, a marker M2 that can be used as an AR marker can also be disposed on a housing H of an ultrasound probe 1A. The marker M2 is different from the marker M1 disposed on the projection target member 4 and the reference figure projected toward the projection target member 4 by the reference figure projection light source 11. In a case where the ultrasound probe 1A is within the visual field of the optical camera 3, the probe position estimation unit 24 can estimate a position and an orientation of the ultrasound probe 1A by taking into account the marker M2 disposed on the housing H of the ultrasound probe 1A in addition to the image R1 of the reference figure projected onto the projection target member 4. Accordingly, the accuracy of estimating the position and the orientation of the ultrasound probe 1A can be improved.

In addition, although an example in which the marker M1 disposed on the projection target member 4, the reference figure projected from the reference figure projection light source 11 onto the projection target member 4, and the marker M2 disposed on the ultrasound probe 1A are formed of white and black patterns is described, the colors are not particularly limited, and these can also be formed of patterns having colors other than white and black.

### Second Embodiment

In the first embodiment, the position and the orientation of the ultrasound probe 1 are always estimated by using the image R1 of the reference figure projected onto the projection target member 4, but the position and the orientation of the ultrasound probe 1 can also be estimated using the image R1 of the reference figure only in a case where the ultrasound probe 1 is in a blind spot of the visual field of the optical camera 3.

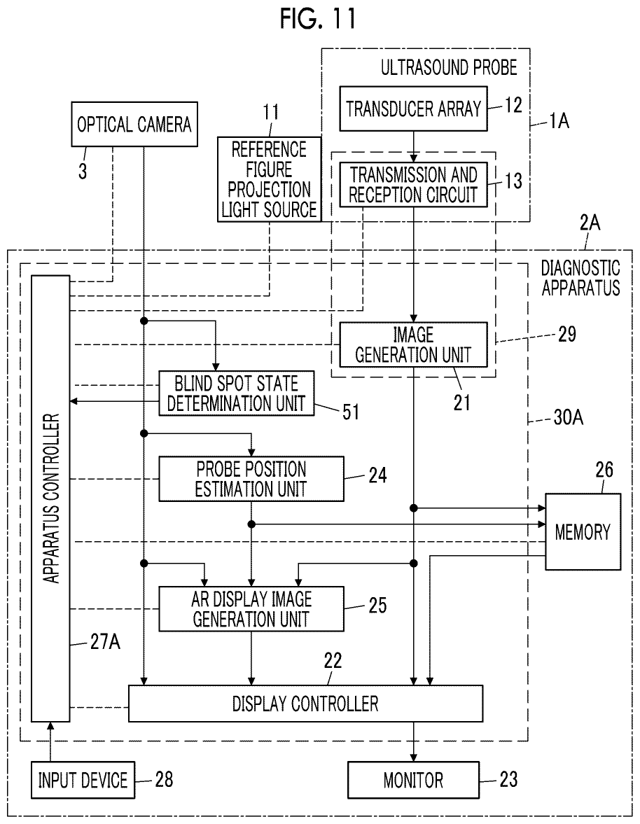

FIG. 11 illustrates a configuration of the ultrasound probe 1A and a diagnostic apparatus 2A in the second embodiment. The ultrasound probe 1A is the same as the ultrasound probe 1 in the first embodiment except that the marker M2 is disposed on the housing H as illustrated in FIG. 10.

The diagnostic apparatus 2A further comprises a blind spot state determination unit 51 in the diagnostic apparatus 2 in the first embodiment illustrated in FIG. 2 and comprises an apparatus controller 27A instead of the apparatus controller 27. In the diagnostic apparatus 2A, the blind spot state determination unit 51 is connected to the optical camera 3. The blind spot state determination unit 51 is connected to the apparatus controller 27A. The reference figure projection light source 11 is connected to the apparatus controller 27A. Further, the image generation unit 21, the display controller 22, the probe position estimation unit 24, the AR display image generation unit 25, the apparatus controller 27A, and the blind spot state determination unit 51 constitute a processor 30A for the diagnostic apparatus 2A.

The blind spot state determination unit 51 determines whether or not the marker M2 of the ultrasound probe 1A enters the blind spot of the visual field of the optical camera 3 by analyzing the optical image Q acquired by the optical camera 3. The blind spot state determination unit 51 performs processing of reading the marker M2 of the ultrasound probe 1A from the optical image Q, for example, in the same manner as the probe position estimation unit 24, and can determine that the ultrasound probe 1A has entered the blind spot of the visual field of the optical camera 3 in a case where a state in which the marker M2 installed on the ultrasound probe 1A cannot be read from the optical image Q continues for a predetermined time or longer.

The reference figure projection light source 11 stops the projection of the reference figure with respect to the projection target member 4 in a case where the blind spot state determination unit 51 determines that the marker M2 is located outside the blind spot of the visual field of the optical camera 3, that is, within the visual field of the optical camera 3, and starts the projection of the reference figure with respect to the projection target member 4 in a case where the blind spot state determination unit 51 determines that the marker M2 has entered the blind spot of the visual field of the optical camera 3, under the control of the apparatus controller 27A.

The probe position estimation unit 24 estimates the position and the orientation of the ultrasound probe 1A with respect to the optical camera 3 based on the marker M2 of the ultrasound probe 1A while it is determined that the marker M2 of the ultrasound probe 1A is located within the visual field of the optical camera 3. Further, the probe position estimation unit 24 estimates the position and the orientation of the ultrasound probe 1A based on the image R1 of the reference figure projected from the reference figure projection light source 11 onto the projection target member 4 while it is determined that the marker M2 of the ultrasound probe 1A is in the blind spot of the visual field of the optical camera 3.

As described above, according to the ultrasound diagnostic system of the second embodiment, even in a case where the position and the orientation of the ultrasound probe 1A are estimated based on the image R1 of the reference figure projected onto the projection target member 4 only in a case where the ultrasound probe 1A is in the blind spot of the visual field of the optical camera 3, the position and the orientation of the ultrasound probe 1A with respect to the optical camera 3 can be estimated regardless of whether or not the ultrasound probe 1A is in the blind spot of the visual field of the optical camera 3.

### EXPLANATION OF REFERENCES

1, 1A: ultrasound probe
2, 2A: diagnostic apparatus
3: optical camera
4: projection target member
11: reference figure projection light source
12: transducer array
13: transmission and reception circuit
21: image generation unit
22: display controller
23: monitor
24: probe position estimation unit
25: AR display image generation unit
26: memory
27, 27A: apparatus controller
28: input device
29: image acquisition unit
30, 30A: processor
41: pulser
42: amplifying unit
43: AD conversion unit
44: beam former
45: signal processing unit
46: DSC
47: image processing unit
51: blind spot state determination unit
A1: projection target region
CW: connection cable
D: direction vector
E: AR display image
H: housing
H1: array accommodation portion
H2: grip portion
H2A: tip portion
J: hand
M1, M2: marker
Q: optical image
R1: image
U: ultrasound image

What is claimed is:

1. An ultrasound diagnostic system comprising:
an ultrasound probe having a light source that projects a reference figure;
a projection target member onto which an image of the reference figure is projected from the light source;
a diagnostic apparatus connected to the ultrasound probe; and
an optical camera that is connected to the diagnostic apparatus and is configured to acquire an optical image in which the projection target member appears,
wherein the diagnostic apparatus includes a processor, and
the processor is configured to estimate a position and an orientation of the ultrasound probe based on the image of the reference figure projected onto the projection target member that appears in the optical image.

2. The ultrasound diagnostic system according to claim 1,
wherein a marker is installed on the ultrasound probe, and
the processor is configured to estimate the position and the orientation of the ultrasound probe by taking into account the marker installed on the ultrasound probe that appears in the optical image.

3. The ultrasound diagnostic system according to claim 2,
wherein the processor is configured to:
determine whether or not the marker of the ultrasound probe enters a blind spot of a visual field of the optical camera by analyzing the optical image acquired by the optical camera; and
estimate the position and the orientation of the ultrasound probe based only on the image of the reference figure projected onto the projection target member while determining that the marker has entered the blind spot of the optical camera.

4. The ultrasound diagnostic system according to claim 3,
wherein the processor is configured to determine that a state in which the marker installed on the ultrasound probe is not read from the optical image continues for a predetermined time or longer, as that the ultrasound probe has entered the blind spot of the optical camera.

5. The ultrasound diagnostic system according to claim 3,
wherein upon determining by the processor that the marker is located outside the blind spot of the optical camera, the projection of the reference figure onto the projection target member by the light source is stopped, and
upon determining by the processor that the marker has entered the blind spot of the optical camera, the projection of the reference figure onto the projection target member by the light source is started.

6. The ultrasound diagnostic system according to claim 4,
wherein upon determining by the processor that the marker is located outside the blind spot of the optical camera, the projection of the reference figure onto the projection target member by the light source is stopped, and
upon determining by the processor that the marker has entered the blind spot of the optical camera, the projection of the reference figure onto the projection target member by the light source is started.

7. The ultrasound diagnostic system according to claim 1, further comprising:
a memory,
wherein the processor is configured to:
acquire an ultrasound image of a subject by transmitting and receiving an ultrasound beam using the ultrasound probe; and
store, in the memory, information on the estimated position and orientation of the ultrasound probe and the ultrasound image in association with each other.

8. The ultrasound diagnostic system according to claim 2, further comprising:
a memory,
wherein the processor is configured to:
acquire an ultrasound image of a subject by transmitting and receiving an ultrasound beam using the ultrasound probe; and
store, in the memory, information on the estimated position and orientation of the ultrasound probe and the ultrasound image in association with each other.

9. The ultrasound diagnostic system according to claim 3, further comprising:
a memory,
wherein the processor is configured to:
acquire an ultrasound image of a subject by transmitting and receiving an ultrasound beam using the ultrasound probe; and
store, in the memory, information on the estimated position and orientation of the ultrasound probe and the ultrasound image in association with each other.

10. The ultrasound diagnostic system according to claim 4, further comprising:
a memory,
wherein the processor is configured to:
acquire an ultrasound image of a subject by transmitting and receiving an ultrasound beam using the ultrasound probe; and
store, in the memory, information on the estimated position and orientation of the ultrasound probe and the ultrasound image in association with each other.

11. The ultrasound diagnostic system according to claim 5, further comprising:
a memory,
wherein the processor is configured to:
acquire an ultrasound image of a subject by transmitting and receiving an ultrasound beam using the ultrasound probe; and
store, in the memory, information on the estimated position and orientation of the ultrasound probe and the ultrasound image in association with each other.

12. The ultrasound diagnostic system according to claim 6, further comprising:
a memory,
wherein the processor is configured to:
acquire an ultrasound image of a subject by transmitting and receiving an ultrasound beam using the ultrasound probe; and
store, in the memory, information on the estimated position and orientation of the ultrasound probe and the ultrasound image in association with each other.

13. A control method of an ultrasound diagnostic system, the control method comprising:
projecting a reference figure onto a projection target member from an ultrasound probe;
acquiring an optical image in which the projection target member appears by using an optical camera; and
estimating a position and an orientation of the ultrasound probe based on an image of the reference figure projected onto the projection target member that appears in the optical image.
